(19) United States
(12) Patent Application Publication
(10) Pub. No.: US 2010/0233267 A1
(43) Pub. Date: Sep. 16, 2010

(54) COMPOSITE HYDROGEL

(75) Inventors: Weiliam Chen, Mount Sinai, NY (US); Lihui Weng, Ames, IA (US); Hui Pan, Stony Brook, NY (US)

Correspondence Address: FISH & RICHARDSON PC, P.O. BOX 1022, MINNEAPOLIS, MN, 55440-1022, US

(73) Assignee: The Research Foundation of State University of New York, Albany, NY (US)

(21) Appl. No.: 12/525,513

(22) PCT Filed: Feb. 1, 2008
(86) PCT No.: PCT/US2008/052805

§ 371 Date: May 24, 2010

Related U.S. Application Data
(60) Provisional application No. 60/887,796, filed on Feb. 1, 2007.

(51) Int. Cl. A61K 9/14; A61K 35/12; A61K 39/395; A61K 38/16; A61K 31/7088; A61K 38/28

(52) U.S. Cl. 424/488; 424/93.7; 424/130.1; 514/12; 514/44 R; 514/44 A; 514/3

(57) ABSTRACT

The present invention features a composite hydrogel for use as soft tissue substitutes and transitional three-dimensional support structures.

### CLAIM OF PRIORITY

This application claims the benefit of U.S. Provisional Patent Application Ser. No. 60/887,796, filed on Feb. 1, 2007, the entire contents of which is hereby incorporated by reference.

### FEDERALLY SPONSORED RESEARCH OR DEVELOPMENT

This invention was made with Government support awarded by the National Institutes of Diabetes and Digestive Kidney Diseases under Campus No. 1041956-1-33476: Sponsor No. 5R01DK068401-02. The Government has certain rights in the invention.

### TECHNICAL FIELD

The present invention relates to wound healing and tissue engineering, and more particularly to methods of making composite hydrogels for use as soft tissue substitutes and transitional three-dimensional support structures to enhance chronic wound repair and support tissue regeneration and reconstruction.

### BACKGROUND

Wound healing involves a series of highly coordinated cellular events that result in the architectural and functional restoration of damaged of tissue. In the case of chronic wounds, however, the healing process is impeded and tissue restoration is delayed.

About 6% of the US population have diabetes, and one of the most serious complications of diabetes is the development of chronic non-healing diabetic foot ulcers. Currently, 3% of the diabetic population develop foot ulcers per year, and 15% of all diabetics experience at least one episode during their life. Without appropriate prophylactic risk management to prevent or delay the formation of these injuries, and in the absence of any truly effective therapeutic agents, the only option available for the treatment of chronic non-healing wounds is frequently surgical amputation. As a result, 95% of the 74,000 lower extremity amputations performed in the US in 1996 were attributable to diabetes.

### SUMMARY

In one aspect, the present invention features methods for creating a self-crosslinkable hydrogel containing hyaluronan (e.g., a partially oxidized hyaluronan) and gelatin. The methods can include the steps of providing a first solution containing a partially oxidized hyaluronan and a second solution containing gelatin. One or more of the components of the first or second solution may be obtained from (e.g., isolated from) a human source. The first and second solutions are mixed to create a third solution in which a self-crosslinking reaction occurs to form a partially oxidized hyaluronan and gelatin-containing hydrogel. The amounts of hyaluronan and gelatin in the hydrogel can vary. For example, the hyaluronan and gelatin can be present at a ratio of about 5:5; a ratio of about 4:6; or a ratio of about 6:4. While the compositions of the invention are not limited to those in which hyaluronan and gelatin associate with one another in any particular way, they may include those compositions formed when hyaluronan (e.g., partially oxidized hyaluronan) and gelatin are chemically crosslinked by way of Schiff base formation between the aldehyde groups in the partially oxidized hyaluronan and the ε-amino group of a lysine or hydroxylysine residue of gelatin.

The hydrogel can include additional agents. For example, in one aspect, the hydrogel can include one or more types of biological cells (e.g., genetically engineered biological cells). For example, the hydrogel can contain a connective tissue cell, a fibroblast, an epithelial cell, an epidermal or dermal cell, a chondrocyte, an osteocyte (e.g., an osteoblast), a blood or plasma cell, an adipocyte, a myocyte, a hepatocyte, a neuron, a glial cell, an endocrine cell (e.g., an islet cell), a cell of a sensory organ, or a mesenchymal cell. The hydrogel may also contain a stem cell or a partially differentiated progenitor cell.

Alternatively, or in addition, the hydrogel can include a bioactive agent, such as a pharmaceutical agent, a growth factor, or a component of the extracellular matrix (e.g., collagen). A hydrogel containing a bioactive agent may be used as a vehicle to deliver, with controllable kinetics, a bioactive agent into a patient's tissue. Suitable bioactive agents include one or more of: a therapeutic antibody, a toxin, a chemotherapeutic agent, an anti-angiogenic agent, insulin or other hormone, an antibiotic, an analgesic or anesthetic agent, an antiviral agent, an anti-inflammatory agent, an antithrombolytic agent, an RNA that mediates RNA interference, a microRNA, an aptamer, a peptide or peptidomimetic, or an immunosuppressant.

Native HA or materials like carboxymethylcellulose can be added to the partially oxidized hyaluronan/gelatin blend as a material property modifier.

Dyes such as an MRI-appropriate dye (e.g., gadolinium-albumin) or other radioopaque or fluorescent markers can also be included.

The present hydrogels may have a Poisson's ratio of between about 0.40 and 0.80.

In an alternative embodiment, the hydrogel may be combined with a medical device for the treatment of both external or internal wounds. Medical devices that may accommodate the hydrogel include dressings for wounds, vascular stents, orthopedic devices, and drug delivery devices.

The hydrogels may be used for tissue augmentation or soft tissue repair. Such methods of treatment can include a step of identifying a suitable patient (i.e., a patient who would benefit from tissue augmentation or repair). The hydrogels would be suitable for tissue augmentation or repair in cases in which the soft tissue is sub-epithelial tissue, cartilage, liver, or neural tissue within the central or peripheral nervous system. Further applications for the hydrogels of the invention may include the treatment and repair of sub-epithelial dermal tissue that has been damaged by trauma. The hydrogels may also be particularly useful for the augmentation or repair of sub-epithelial dermal tissue damaged by disease, more specifically, diabetes.

In an alternative embodiment, the invention features kits containing the hydrogel or components to make the hydrogel (e.g., the solutions described above), and instructions for use in, for example, any of the circumstances described herein.

In final embodiment, the hydrogel can be substantially free of a chemoattractant.

The present hydrogels may provide superior systems that foster cell infiltration and promote cell viability. Upon implantation, the hydrogels may serve as transient support structures that mimic the ECM and thereby facilitate tissue regeneration.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagrammatic representation of (A) oxidation of hyaluronan (HA) by sodium periodate, and (B) a crosslinking reaction between partially oxidized HA (oHA) and gelatin.

FIG. 2 is a FT-IR spectra of (A) HA, (B) oHA, (C) oHA/gelatin hydrogel, and (D) gelatin.

FIG. 3 is a scanning electron micrograph (SEM) of oHA with an oxidation degree of 27.8 (A) oHG-7, (B) oHG-4, (C) oHG-6, and (D) 20% by weight gelatin hydrogel. Scale bar is 10 μm.

FIG. 4 is a line graph showing the correlation of swelling ratio (q) of oHA/gelatin hydrogels on the oxidation degree of oHA.

FIG. 5 is a line graph showing the correlation of storage modulus G′ on the oscillating frequency of oHA/gelatin hydrogels.

FIG. 6 is a line graph showing the correlation of storage modulus G′ and loss modulus G″ on oscillatory shear stress of oHA/gelatin hydrogels.

FIG. 7 is a schematic representation of contact assays: (A) direct, and (B) indirect.

FIG. 8 is an image demonstrating cell attachment, distribution, and proliferation in oHA/gelatin hydrogel (oHG-6). Cross-sections of cell-laden hydrogel: (A-B)3 days; 200× magnification, (C-D) 5 days; 100× magnification, and (E-F) after cell seeding; 400× magnification. Samples were stained with crystal violet (A,C, and E) and Live/Dead dye (B, D, and F), respectively. Demarcation (S), hydrogel surface (I), hydrogel interior (F), fibroblasts (I), dead cells (red in the original color photograph) and live cells (green in the original color photograph).

FIG. 9 is a bar graph showing MTS assay of cell viability on various oHA/gelatin hydrogel formulations.

FIG. 10 is a SEM of the in vitro deposition of ECM and cell-mediated degradation of oHG-6 hydrogel. (A-B) Pores of hydrogel were masked and filled with ECM at day 5 after cell seeding; (A) surface, and (B) cross-section. Tunnel created by cell infiltration; (C) interior of hydrogel where cells did not reach remained intact. (D) morphology of hydrogel showing degradation.

FIG. 11 is a bar graph showing the cell mediated degradation time of oHG-1, oHG-2, and oHG-6 hydrogels.

FIG. 12 is an image demonstrating H&E staining of explanted hydrogel. (A-B) day 3 post-implantation. (C-D) day 7 post-implantation. (C) thin fibrous capsule. (H) hydrogel; (↑) macrophage; (↓) Fibroblast; (E-F) day 21 post-implantation

FIG. 13 is a SEM of the in vivo deposition of ECM and cell-mediated degradation of oHG-6 hydrogel. Extensive ECM deposition on the surface (A), and cross-section (B) of explanted hydrogels 1 week post-implantation. (*) tunnel created by cell infiltration.

### DETAILED DESCRIPTION

The present invention features a method for creating a self-crosslinkable hydrogel containing partially oxidized Hyaluronan (oHA) and gelatin. Hyaluronan (HA), also referred to by those in the art as hyaluronic acid or hyaluronate, is a naturally occurring, high molecular weight, non-sulfated glycosaminoglycan synthesized in the plasma membrane of fibroblasts and other cells. HA is one of several glycosaminoglycans that are widely distributed around the body. HA is a universal component of the extracellular matrix (ECM) and is also found concentrated throughout connective, epithelial, and neural tissue. A 70 kg male has on average 15 g of HA, one third of which is degraded and synthesized daily.

HA is a linear polysaccharide composed of repeating disaccharides, which themselves are composed of D-glucuronic acid and D-N-acetylglucosamine linked together by alternating β-1, 4 and β-1, 3 glycosidic bonds. Polymers of HA range in size from 1×10⁵ to 5×10⁶ Daltons, however, are most frequently towards the higher end of this range. The structure of HA is homologous in all species and it is immunologically inert. These unique attributes make this polysaccharide and ideal substance for use as a biomaterial in health and medicine.

HA is available commercially from a number of manufacturers. The most commonly used form is non-animal stabilized hyaluronic acid (NASHA), produced by bacterial fermentation from streptococci bacteria. As NASHA is derived from a non-animal source, its use further reduces the risk of immunogenicity and disease transmission.

Hydrogels are highly porous biomaterials that permit gas nutrient exchange, facilitating long term cell survival. Potential applications for hydrogels include soft tissue substitutes in tissue engineering and chronic wound healing. An important criterion for developing biomedical materials is to mimic the ECM. HA, as a major component of the ECM, therefore, represents an excellent candidate biomaterial.

HA can be stabilized by crosslinking to form hydrogels. Many crosslinking agents, however, have cytotoxic potential. Furthermore, the β-1, 4 backbone linkage and repeat pyranoid ring structure render HA inherently brittle. Consequently, it is difficult to preserve the structural integrity of HA in a hydrogel.

Partial oxidation of HA, using sodium periodate (NaIO₄), is a strategy to circumvent the rigidity of HA by introducing open rings into the structure of native HA, thereby enhancing its elasticity. Sodium periodate is commonly used to open saccharide rings between vicinal diols, leaving two aldehyde groups. Exposure of HA to sodium periodate oxidizes the proximal groups of HA to aldehyde; correspondingly, oxidation opens the glucose ring to form a linear chain and the breakage of each C—C bond produces two aldehyde groups. The oxidization degree of HA can be controlled by adjusting the feed ratio of HA (purchased from Englehard Inc., Stony Brook, N.Y.) to sodium periodate (Sigma-Aldrich, St. Louis, Mo.) in a reaction.

Gelatin is a widely commercially available natural polymer derived from collagen with unique gelation properties, which like HA, is abundant in the ECM. Gelatin is a liquid at room temperature above its gelation temperature while a gel below; this is a result of the physical crosslinking attributable to partial recovering of the triple-helix conformation of native gelatin. Gelatins potential in biomedical applications are limited as gelatin is very brittle and cannot retain its shape within body temperature range. Chemical crosslinking using Schiff base formation between the ε-amino groups of lysine or hydroxylysine side groups of gelatin and the aldehyde groups of crosslinkers, such as HA, is a strategy to circumvent this limitation.

Partially oxidized HA and gelatin (oHA/Gelatin) hydrogels were prepared by blending oHA with varying degrees of oxidaton with gelatin derived from a human source (Purchased from Sigma-Aldrich, St. Louis, Mo.) using ratios of about 5:5, about 4:6, and about 6:4. Tetraborate decahydrate (borax) was including in the self-crosslinking reaction due to its widely reported ability to enhance the solubility of polysaccharides and provide the correct pH for Schiff bond formation. As both HA and gelatin are the major structural components of the ECM hydrogels composed of these materials could be excellent biomaterials for use in health and medicine. In addition, oHA/Gelatin hydrogel formation does not require the addition of any chemical crosslinking agents, the chance of cytotoxic effects are minimal.

In another aspect, the present invention will combined with one or more biological cells to further enhance wound healing of specific tissues. For example, dermal fibroblast cells may be incorporated to treat chronic non healing wounds.

The present invention may also be adapted to incorporate a bioactive agent to treat a certain disease state, such as a pharmaceutical agent or a component of the ECM. Candidate pharmaceutical agents, could include but are limited to, a therapeutic antibody, an analgesic, an anesthetic, an antiviral agent, an anti-inflammatory agent, an RNA that mediates RNA interference, a microRNA, an aptamer, a peptide or peptidomimetic, an immunosuppressant, hypoxyapatite, or bioglass. The above bioactive agents could be released acutely or via a slow release mechanism.

The hydrogels may also be combined with medical devices for the treatment of both external or internal wounds. The hydrogels may be applied to bandages for dressing external wounds, such as chronic non-healing wounds, or used as subdermal implants. Alternatively, the present hydrogels may be used in organ transplantation, such as live donor liver transplantation, to encourage tissue regeneration. The hydrogels may be adapted to individual tissue types by equilibrating the water content, biodegradation kinetics, and Poisson's ratio with those of the target tissue to be repaired.

An alternative embodiment for the present invention is for utilization in the field of tissue engineering and regeneration. The hydrogels may serve as transient three-dimensional scaffolds and may mimic the ECM and support cell infiltration, viability, and tissue regeneration. One can equilibrate the water content, biodegradation kinetics, and Poisson's ratio with that of the tissue to be regenerated.

The hydrogels may also be applied for tissue augmentation or soft tissue repair. As described above, the hydrogels may be tailored for an individual or tissue type in need of augmentation or repair. The hydrogels would be suitable for tissue augmentation or repair in cases in which the soft tissue is sub-epithelial tissue, cartilage, liver, or neural tissue within the central or peripheral nervous system. Further applications for the hydrogels may include the treatment and repair of sub-epithelial dermal tissue that has been damaged by trauma or that would benefit cosmetically. The hydrogels may also be particularly useful for the augmentation or repair of sub-epithelial dermal tissue damaged by disease, more specifically, diabetes.

In a final embodiment, the invention features a kit containing the hydrogel intended for use in any of the aforementioned features or solutions that, when combined, form a hydrogel. That is, the kits may contain the hydrogels in a pre-made state or individual components thereof to be prepared prior to use. The components of the kits can be tailored for specific applications, including one or more of the following. Cell delivery in which the invention would be supplied containing one or more of the following cells. fibroblast, an epithelial cell, an epidermal or dermal cell, a chondrocyte, an ostreocyte, a blood or plasma cell, an adipocyte, or a mesenchymal cell. The hydrogel may also contain a stem cell or a partially differentiated progenitor cell.

When fashioned as a drug delivery vehicle, the hydrogel can contain one or more of the following bioactive agents: a therapeutic antibody, an analgesic, an anesthetic, an antiviral agent, an anti-inflammatory agent, an RNA that mediates RNA interference, a microRNA, an aptamer, a peptide or peptidomimetic, an immunosuppressant, hypoxyapatite, or bioglass. The hydrogels may also be supplied with or already applied to medical devices or dressings.

### EXAMPLES

### Example 1

### Preparation of Partially Oxidized Hyaluronan (oHA)

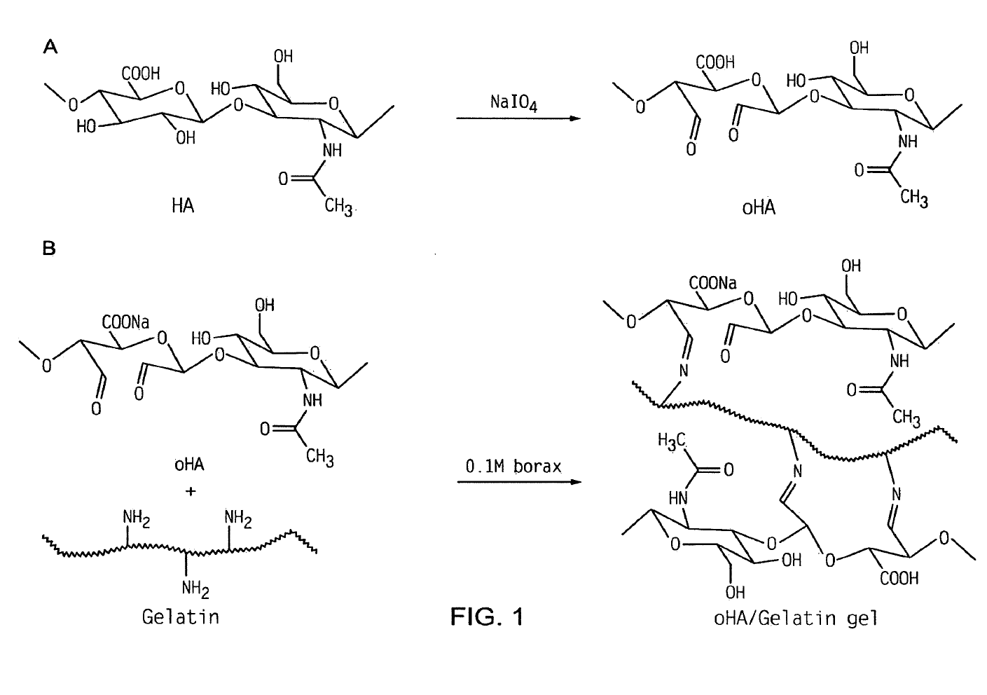

The partial oxidation of Hyaluronan (HA) is shown schematically in FIG. 1A. In a typical preparation, one gram of sodium HA was dissolved in 80 ml of water in a flask shaded by aluminum foil. Partial oxidization of HA was driven using varying amounts of sodium periodate (o-periodate. NaIO₄), which was dissolved in 20 ml of water and added drop wise to the sodium HA solution. HA oxidization was allowed to proceed at an ambient temperature for a stipulated period of time before adding 10 ml of ethylene glycol to terminate the reaction. Solutions were subsequently stirred at room temperature for 1 hour and extensively dialyzed against water for three days. The resulting product was pure partially oxidized Hyaluronan (oHA) with a yield of 50-67%.

The degree of HA oxidization was manipulated by varying the sodium periodate to HA ratio in the reaction. HA oxidation was then assessed by quantifying total aldehyde residue—formed by partial oxidation—content in oHA. To avoid misinterpretation caused by aldehyde residues assuming hemiacetal conformations, which would not be directly detected by ¹H NMR, aldehyde groups on oHA were reacted with excess tert-butyl carbazate. Briefly, a pH 5.2 acetate buffer was prepared containing 10 mg/ml oHA. A 5-fold excess of tert-butyl carbazate was then added to the same buffer and the reaction was allowed to proceed for 24 hours at ambient temperature. During this incubation, aldehyde residues on the oHA formed C═N bonds. These C═N bonds were subsequently reduced to C—N by adding a 5-fold excess of NaBH₃CN to the reaction and incubating for a further 12 hours. The final reaction product was then precipitated three times with acetone, dialyzed against water, and lyophilized. The degree of oxidation, or abundance of aldehyde groups, was then determined using ¹H NMR, and a summary of this data is presented in Table 1. As shown in Table 1, the oxidation degree of HA (experimental oxidation degree) varied significantly (16.7% to 57.8%) when the ratio of sodium periodate to HA (theoretical oxidation degree) varied from 20% to 70%.

Mean molecular weights were determined using HPLC and polyethylene glycol calibration standards. As shown in Table 1, the mean molecular weight of oHA (Mn) decreased as the ratio of sodium periodate to HA increased. Although not shown, a gradual decrease in the viscosity of oHA solutions indicatives that this may be caused by HA degradation.

TABLE 1
Oxidation Degrees of HA

| | Theoretical oxidation | Experimental Oxidation | Mn |
|---|---|---|---|
| Preparation | Degree (%) | Degree (%) | (kDa) |
| 1 | 20 | 16.7 | 183.8 |
| 2 | 30 | 20.3 | 71.1 |
| 3 | 40 | 57.8 | 57.8 |
| 4 | 50 | 41.2 | 41.2 |
| 5 | 70 | 35.5 | 35.5 |

### Example 2

### Formation of a HA and Gelatin Hydrogel

The formation of a oHA and gelatin (oHA/Gelatin) hydrogel is shown schematically in FIG. 1B. Briefly, 20% (w/v) solutions of oHA and gelatin were prepared separately in a buffer containing 0.1 M tetraborate decahydrate (borax) at pH 9.4. Hydrogel formation was initiated by mixing oHA and gelatin solutions using weight ratios of about 5:5, 4:6, and 6:4 (see Table 2). Solutions were then gently stirred for 1 minute at 37° C. and incubated at 37° C. for up to 12 hours. Hydrogels were stored at 5° C. until utilization. oHA/Gelatin hydrogels were prepared using oHA with different oxidation degrees, as summarized in Table 2.

TABLE 2
Formulations of oHA/Gelatin Hydrogels

| Preparation | Oxidation Degree of oHA (%) | oHA:Gelatin |
|---|---|---|
| oHG-1 | 16.7 | 5:5 |
| oHG-2 | 20.3 | 5:5 |
| oHG-3 | 23.4 | 5:5 |
| oHG-4 | 27.8 | 5:5 |
| oHG-5 | 44.4 | 5:5 |
| oHG-6 | 27.8 | 4:6 |
| oHG-7 | 27.8 | 6:4 |

### Example 3

### Characterization of oHA and oHA/Gelatin Hydrogels

Infrared spectra of HA, oHA, oHA/Gelatin hydrogels, and gelatin were recorded using a Galaxy Series Fourier Transformed Infrared (FTIR) 3000 spectrometer. FTIR is a technique commonly used to identify discrete functional groups within a molecule, and is based on the specific and highly consistent infrared adsorption and vibration characteristics of individual functional groups.

FTIR samples were lyophilized, mixed with KBr, and pressed into pellets. All spectra represent an average of 64 scans with a resolution of 4 cm⁻¹.

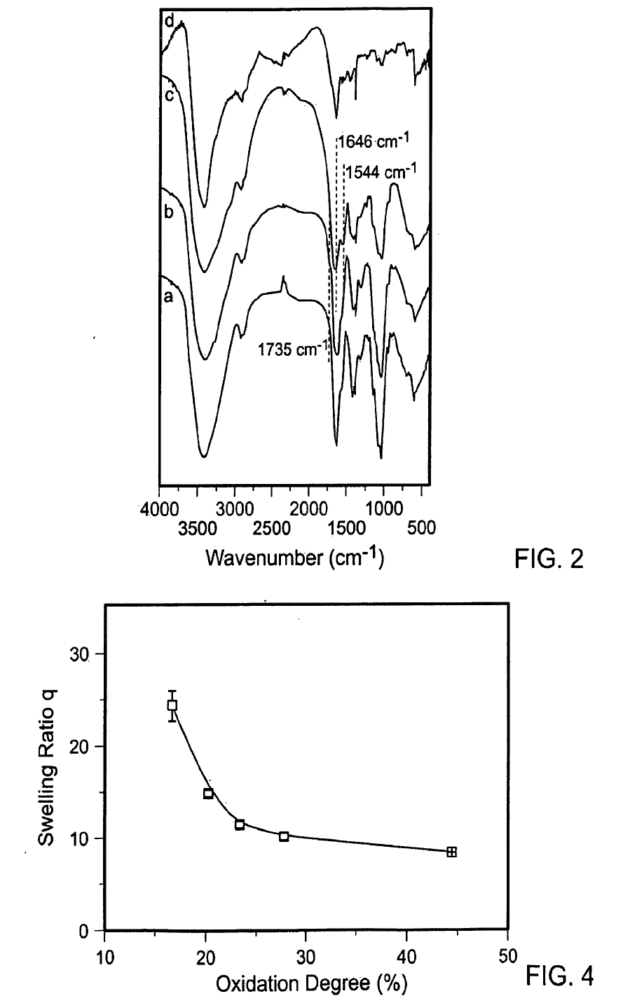

As shown in FIG. 2, HA (a), oHA (b), partially oHA/gelatin hydrogels (c), and gelatin (d) present distinct spectra. In comparison with HA (a), a distinct shoulder at wavenumber 1735 cm⁻¹ was detected in the oHA spectra (b), which is characteristic of the symmetric vibration of aldehyde groups, and further proof that HA in this sample is indeed partially oxidized. As illustrated in FIG. 1B, C═N bonds are established when oHA and gelatin are crosslinked during hydrogel formation. Adsorption peaks detected at 1646 cm⁻¹ and 1544 cm⁻¹ are characteristic of the stretching vibration of C═N bonds and are unequivocal indications that crosslinking occurred between partially oHA and gelatin.

### Example 4

### Morphological Analysis of Hydrogels

The morphological characteristics of oHG-4, oHG-6, and oHG-7 (see Table 1) were examined using Scanning Electronic Microscopy (SEM). In preparation for analysis, oHA/Gelatin hydrogels were snap frozen in a glass container using liquid nitrogen, and lyophilized. Fractured pieces of lyophilized hydrogels 0.5-1.0 cm in length were then secured on an aluminum board using copper tapes. Secured samples were sputtered with gold, and both surface and cross-sectional morphologies were recorded using a field-emission scanning electron microscope at 20 kV.

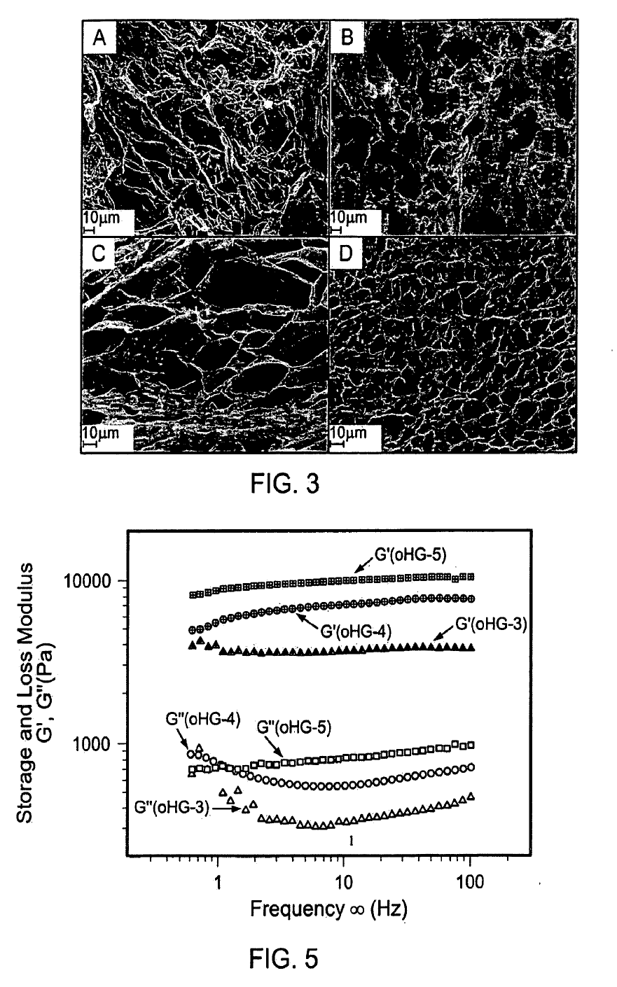

Representative electron micrographs of oHG-7 (A), oHG-4 (B), and oHG-6 (C), and native gelatin (D) are shown in FIG. 3. Clear structural morphological differences were observed on comparison of all hydrogels, irrespective of the oHA to gelatin ratio, with native gelatin. Each hydrogel presented a highly porous morphology and an average pore dimension of 60 μm, which importantly should be accommodative to cell migration. Native gelatin (D), presented a highly porous structure due to the formation of triple helices as crosslinks in the gel: Consequently, increasing gelatin contents resulted in a more porous hydrogel networks with less fibrous structures (compare A and C).

### Example 5

### Hydrogel Swelling Capacity

Oxidation increases the availability of aldehyde residues on HA, and consequently, increases Schiff bond formation with amino groups on gelatin. As Schiff bonds form, amino groups on gelatin are consumed, leading to a reduction in PBS uptake. This principle was applied to analyze the swelling properties of hydrogels.

Swelling studies were performed on oHA/Gelatin hydrogels prepared using oHA with different degrees of oxidation and a constant HA to gelatin ratio of 5:5. The weights of lyophilized hydrogels were recorded (W_{d}) prior to immersion in 0.01 M PBS at 37° C. Following a 48 hour incubation period, hydrogels were blotted to remove excess water and weighed (Wₛ). The swelling ratio (q) was calculated by q=(Wₛ−W_{d})/W_{d}.

As shown in FIG. 4, q decreased approximately 46% when the HA oxidation degree was elevated from 16.7% (oHG-1) to 23.4% (oHG-3). Interestingly, further HA oxidation (above 23.4%) resulted in only moderate changes in q.

The decrease in q observed for oxidation degrees of up to 23.4% is a result of the increase in aldehyde availability caused by oxidation and Schiff bond formation, which as stated above, consumes amino groups on gelatin and reduces PBS uptake, as represented by q. Elevating the hyaluronan oxidation degree beyond 23.4%, however, saturates the amino groups on gelatin, resulting in attenuated PBS uptake and the observed q plateau. This data suggests, therefore, that the majority of amino groups on gelatin are consumed when the HA oxidation degree exceeds 27.8%.

### Example 6

### Rheological Analyses of oHA/Gelatin Hydrogels

Rheological measurements at oscillatory shear deformation of the hydrogels were carried out with a Physica MCR 301 rheometer using parallel plates of 25.0 mm diameter with a plate-to-plate distance of about 2 mm, maintained at constant temperature (25° C.). For frequency sweep tests, the storage modulus G′ and G″ were recorded at a frequency of 1 Hz, with a shear strain of 5%. For shear sweep tests, a constant normal compression force of ˜5 g was applied.

Rheological analyses of the oHA/gelatin hydrogels were performed to quantify their viscoelastic behaviors under periodic strain. The frequency sweeping profiles of oHG-3, OHG-4, and oHG-5 (see Table 2) are depicted in FIG. 5. The loss moduli (G″) weakly depended upon the imposed frequency within the range of 0.5-100 Hz; whereas storage moduli (G′) remained constant. Since the loss moduli were considerably smaller than the storage moduli, the elastic behaviors of the hydrogels dominated their viscous properties, indicating the presence of well-developed networks in oHA/Gelatin hydrogels. The storage modulus increased from 3600 Pa to 10000 Pa when the oxidation degree of oHA was increased from 23.4% to 44.4% (oHG-3 to oHG-5). This could be attributable to the increase in abundance of aldehyde residues leading to more Schiff base formation. Although the magnitude of increase in oxidation degree from 23.4% (oHG-3) to 27.8% (oHG-4) was less than that of from 27.8% (oHG-4) to 44% (oHG-5), the magnitude of corresponding increase in storage moduli from oHG-3 to oHG-4 was considerably greater than from oHG-4 to oHG-5. This suggested the consumption of the bulk of amino groups on gelatin when the oxidation degree reached 27.8% (oHG-4), which was in strong agreement with the results depicted in FIG. 4.

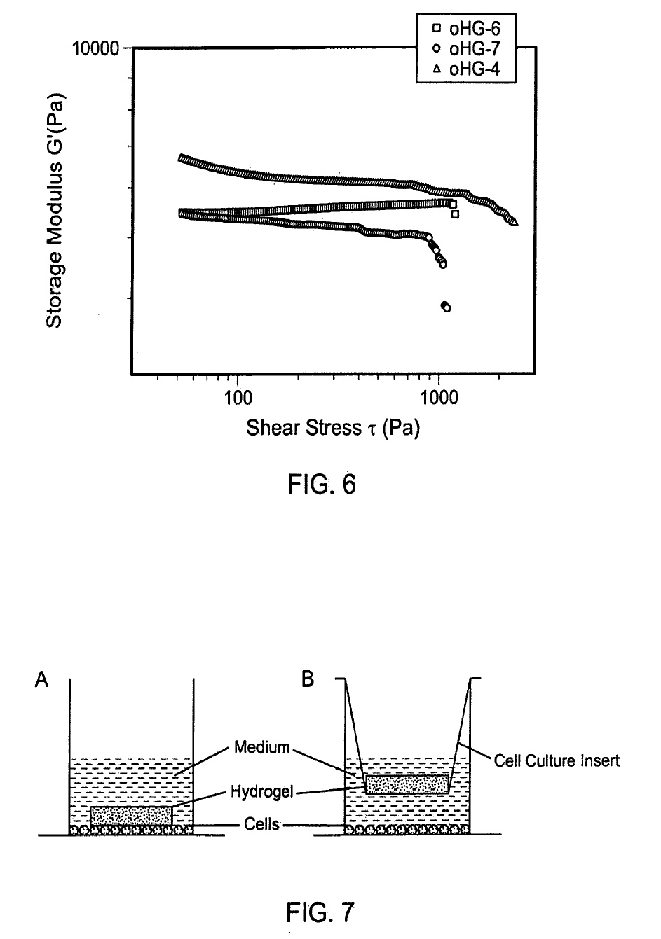

The mechanical behaviors of oHA/gelatin hydrogels were investigated and three typical oscillation stress sweeping profile (oHG-4, oHG-6, and oHG-7) at a frequency of 1 Hz are depicted in FIG. 6. The linear viscoelastic region (LVR) is the stress range where the storage moduli were independent of the applied stress. The storage moduli of oHG-4 and oHG-6 showed a moderate decrease with an increase in the shearing stress due to a slight slipperiness, which occurred when the shear stress was high. The breakdown in shear stress, however, at the end LVR (i.e., the critical stress) was clearly different from the slipperiness-induced decrease in the storage moduli. Among the hydrogel formulations, oHG-4 had the highest storage modulus. Accordingly, the magnitude of the breakdown shear stress of the hydrogels was in the sequence of oHG-4>oHG6.oHG-7, suggesting that the oHA to gelatin ratio was the key contributing factor to the hydrogels mechanical strength. 5:5 appeared to be the optimal ratio for maximizing the reaction of aldehyde and amino groups forming hydrogels with the greatest mechanical strength. Establishing effective inter-chain crosslinks in a hydrogel network could produce a homogeneous and compact hydrogel matrix with a greater degree of elastic response, which corroborated with the SEM results observed in FIG. 3.

### Example 7

### Three-Dimensional Infiltration and Distribution of Cells in Hydrogels

To asses the ability of the oHA/Gelatin hydrogels to support cell infiltration, hydrogels were rinsed extensively in sterile water and PBS before transferring to cell culture medium. As schematically depicted in FIGS. 7 A and B, two independent experimental methods were employed for all hydrogel cell based studies. In the first, direct method (A), hydrogels were in physical contact with the cells. In a second, indirect method (B), hydrogels were placed in polycarbonate cell culture inserts and suspended in the cell culture medium, without direct contact with the cells.

In both systems described above, hydrogels were co-cultured with approximately 200 μL (1×10⁴ cells/mL) of mouse dermal fibroblasts in DMEM (supplemented with 10% fetal bovine serum and 1% Penicillin/streptomycin solution) at 37° C. in a humidified atmosphere of 5% CO₂. Cell culture media were changed daily and cell morphology, adhesion, distribution, viability, proliferation, infiltration, and all histological specimens were observed under an inverted phase contrast light microscope. Images were acquired with Axiovision 4 imaging software.

To analyze cell morphology, attachment, and infiltration into the hydrogels, cell-laden hydrogel samples were retrieved 3 and 5 days post seeding. Cross sections with a thickness of 200 μm were prepared and rinsed twice in PBS, fixed with 70% ethanol for 10 minutes and stained with 0.1% crystal violet (prepared in 200 mM boric acid, pH 8.0) for 5 minutes at ambient temperature. Dye solution was then aspirated and sections rinsed twice with PBS.

Cells attached to all hydrogels, irrespective of HA oxidation degrees or oHA to gelatin ratio within one day of seeding, and notable increases in cell numbers were observed on the surfaces of all hydrogel formulations over the culture span. Once confluent, multiple layers of cells formed on the hydrogel surface. No differences were observed in cell attachment rate or the attachment numbers for any hydrogel formulation.

Analysis of cell infiltration revealed disparities consistent with the crosslinking density of the hydrogel, which in turn was dependent on the oxidation degree of the Hyaluronan. In general, a higher oxidation degree of oHA resulted in a smaller hydrogel pore size. Smaller hydrogel pore size in turn retarded cell infiltration rates. In addition, cell infiltration was affected by oHA to gelatin ratio due to the more distinct pore size caused by a higher gelatin contents: Consequently, hydrogels formulated from a partially oxidized Hyaluronan to gelatin ratio of 4:6 enabled faster cell migration than a hydrogel with a ratio of 5:5.

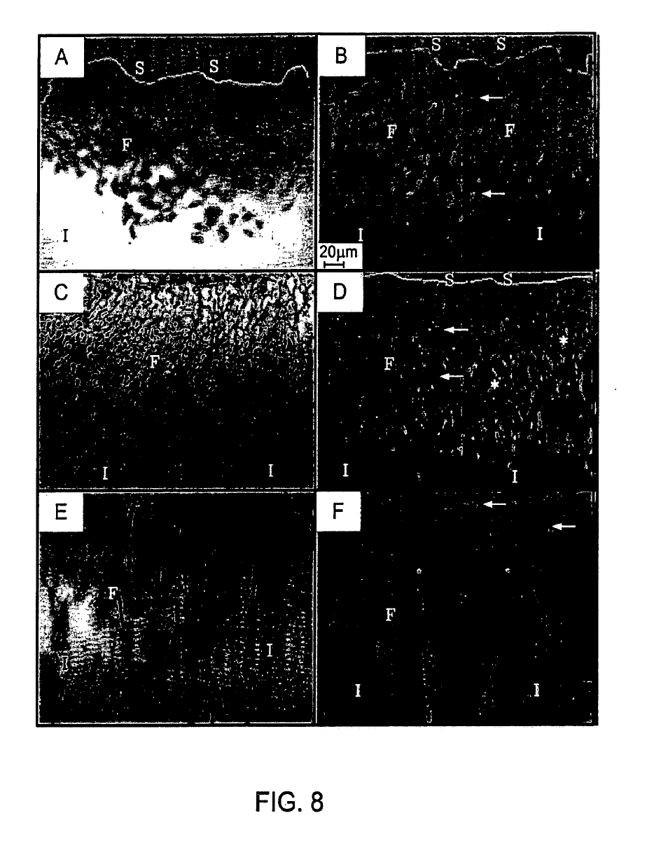

As shown in FIGS. 8 A, C, and E, crystal violet staining revealed that cells infiltrated and distributed evenly throughout oHG-4. Furthermore, cells within the hydrogels presented a highly elongated morphology that was distinctly different to control cells cultured on two-dimensional dishes. Infiltrating cells assumed spherical conformations, consistent with their migration into the hydrogel, and elongated trailing tails, which lined up to form highly organized arrays. Cell numbers and infiltration depth were proportional to the duration of incubation, with the most significant infiltration observed 5 days post seeding (C). Leading cells created channels, which were followed by subsequent trailing cells. A substantial reduction in material cohesiveness was also observed caused by cell-mediated degradation, signifying mechanical deterioration of cell-laden hydrogels.

### Example 8

### Cell Viability in oHA/gelatin Hydrogels

Live/Dead staining assays was performed to evaluate cell viability. Briefly, sections were incubated in 200 μL of Live/Dead dye solution for 10 minutes prior to microscopic analysis using a fluorescent microscope.

As shown in FIGS. 8 B, D, and E, over 99% of cells were alive up to 5 days post seeding (D). Furthermore, the oxidation degree of HA and the ratio of oHA to gelatin did not affect cell viability.

### Example 9

### Long Term Cell Viability in oHA/Gelatin Hydrogels

Cells were cultured for up to 4 weeks using the indirect method described in Example 7 and depicted in FIG. 7B. Hydrogels with approximate dimensions of 5 mm×2 mm×2 mm were collected at 1 week intervals; monolayer cells were used as controls. The MTS assay, which measures mitochondrial activity, was used to determine cell viability.

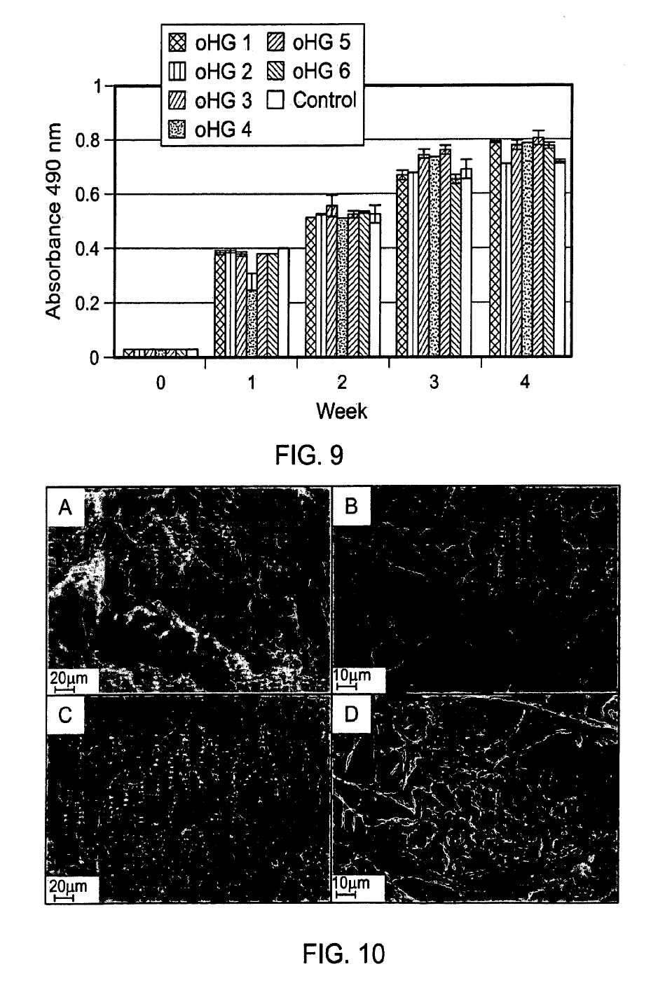

As shown in FIG. 9, over the course of the experiment, total cell numbers increased in all the hydrogel formulations and control samples were confluent by week 3. The absence of any decrease in mitochondrial activity demonstrates that no adverse effects on cell viability were caused by the hydrogel or the degradation products thereof.

### Example 10

### Extracellular Matrix Protein Deposition

Extracellular Matrix (ECM) protein disposition by mouse dermal fibroblast cells was evaluated using SEM and the indirect cell culture method described in Example 7 and depicted in FIG. 7B. Hydrogels were removed from cell culture one month after seeding. Hydrogel surfaces and cross sections were then evaluated for ECM protein deposition.

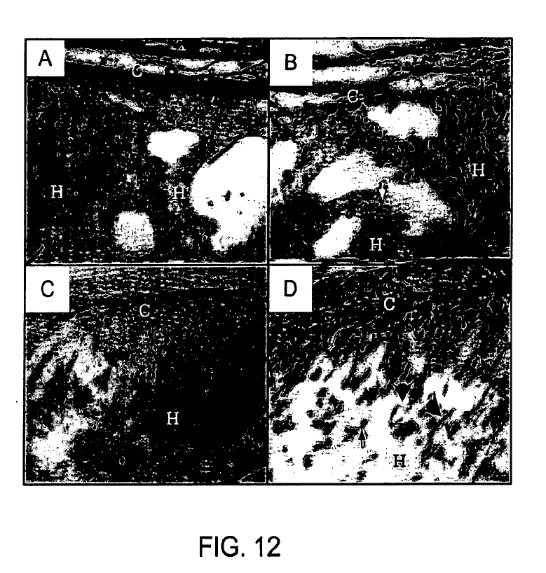

As shown in FIG. 10, abundant ECM protein deposition was observed on the surface (A) and interior (panel B; right hand side) of hydrogels. Furthermore, ECM deposition increased with incubation time. ECM was not observed in regions without cell infiltration (panel B; left hand side); these regions were identical to cell free controls.

### Example 11

### Cell-Mediated Hydrogel Biodegradation

Evaluation of cell-mediated hydrogel degradation by SEM was performed as described in Example 10. Prior to analysis hydrogels were left to undergo degradation by cellular enzymes.

As shown in FIG. 10, the ordered porous structure present in the hydrogel interior (C), not reached by cells, was replaced by a fibrous structure indicative of cell-mediated degradation in infiltrated regions of the hydrogel (D).

Cell-mediated hydrogel degradation kinetics were analyzed using the direct cell culture method described in Example 7 and depicted in FIG. 7A. Hydrogel degradation was defined as the duration from cell seeding until hydrogel disintegration, manifested by a loss in hydrogel cohesiveness.

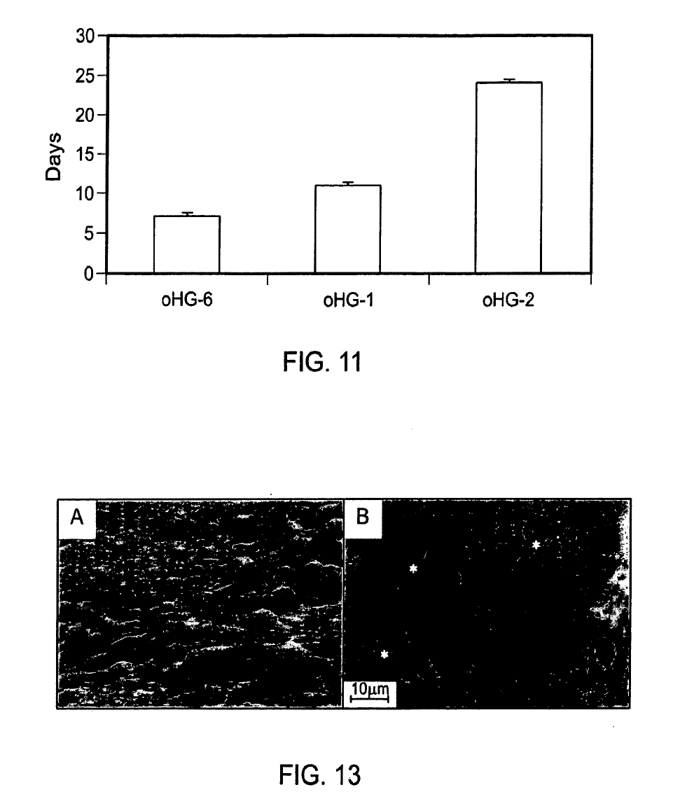

As shown in FIG. 11, hydrogel degradation varied according to the oxidation degree of oHA, which as described above determines the cross linking density of the hydrogel. For hydrogels with a oHA to gelatin ratio of 5:5, the disintegration time increase from 11 to 24 days when the oxidation degree of oHA was increased from 16.7% (oHG-1) to 20.3% (oHG-2). Degradation was not observed within the 30 day time course of this experiment for oHG-3, oHG-4, and oHG-5 (data not shown). Decreasing the amount of oHA, even with an elevated oHA oxidation degree of 27.8, reduced the disintegration time to 7 days (see oHG-6).

### Example 12

### Hydrogel Subdermal Implantation

A mouse subcutaneous implant model was used to evaluate the in vivo biocompatibility and degradation of hydrogels. Hydrogels were sterilized with 70% ethanol followed by extensive rinsing with sterile PBS. Adult female mice were anesthetized using isofluorane and small incisions were made on the dorsal side of each animal. Subdermal pouches were dissected with a blunt probe and two oHG-6 hydrogels were inserted prior to closing the incision. Animals were then euthanized at 3, 7, and 21 day time points, and hydrogels were explanted for histological and SEM evaluation. Explanted implants with surrounding tissue were fixed in 10% neutral buffered formalin. Specimens were cryo-embedded and sectioned with a thickness of 10μm, prior to staining with hematoxylin and eosin (H&E).

Gross examination of the implanted hydrogels revealed an lack of redness or edema, indicating the hydrogels did not evoke an extensive acute inflammatory response, and there was no evidence of tissue necrosis (data not shown). In contrast, tissue in direct contact with the implanted polylactide-co-glycide sutures showed an intense inflammatory response (data not shown).

Considerable hydrogel degradation was observed; 3 days post-implantation hydrogel size was reduced by approximately 50%. One week post-implantation hydrogel size was reduced by 75%. One week post-implantation, hydrogel cohesiveness was consistent with integration with the surrounding host tissue. Three weeks post-implantation, hydrogels were fully resorbed and damaged tissue fully restored. In vitro and in vivo hydrogel degradation rates were highly comparable.

As shown in FIG. 12, consistent with the in vitro data presented in Example 7, histological analysis of the explanted oHG-6 hydrogels revealed extensive cell infiltration. Neutrophils and macrophages were clearly identified in samples collected three days post-implantation (A and B). One week post-implantation, hydrogel implants were encapsulated in a thin, fibrous layer of connective tissue supplied by blood vessels and extensive cell infiltration was observed. Cell density was notably higher towards the hydrogel edge, with cells aligned into highly-organized arrays, comparable to those observed in FIG. 8. The hydrogel interior was populated primarily by fibroblasts, scattered with some macrophages, neutrophils, and mast cells.

As shown in FIG. 13, one week post-implantation considerable ECM protein deposits were observed in the explanted oHG-6 hydrogels (A). Moreover, the porous architectural structure of the hydrogel (B) was barely distinguishable. The massive deposition of ECM is expected to be responsible for the maintained cohesiveness in implanted hydrogels.

1. A hydrogel comprising (a) partially oxidized hyaluronan and (b) gelatin.

2. The hydrogel of claim 1, wherein the hyaluronan and the gelatin are chemically crosslinked.

3. The hydrogel of claim 2, wherein the hyaluronan and the gelatin are chemically crosslinked by way of Schiff base formation.

4. The hydrogel of claim 3, wherein the Schiff base forms between the ε-amino group of a lysine or hydroxylysine residue of gelatin and an aldehyde group in the partially oxidized hyaluronan.

5. The hydrogel of claim 1, wherein the hydrogel further comprises a biological cell.

6. The hydrogel of claim 5, wherein the biological cell is a connective tissue cell.

7. The hydrogel of claim 6, wherein the connective tissue cell is a fibroblast, an epithelial cell, an epidermal or dermal cell, chondrocyte, osteocyte, a blood or plasma cell, a reticular cell, an adipocyte, or a mesenchymal cell.

8. The hydrogel of claim 5, wherein the biological cell is a stem cell or partially differentiated progenitor cell.

9. The hydrogel of claim 1, wherein the hydrogel is substantially free of a chemoattractant.

10. The hydrogel of claim 1, wherein the hydrogel further comprises a bioactive agent.

11. The hydrogel of claim 10, wherein the bioactive agent is a pharmaceutical agent.

12. The hydrogel of claim 11, wherein the pharmaceutical agent is selected from the group consisting of is a therapeutic antibody, a toxin, a chemotherapeutic agent, an anti-angiogenic agent, insulin, an antibiotic, an analgesic or anesthetic agent, an antiviral agent, an anti-inflammatory agent, an antithrombolytic agent, an RNA that mediates RNA interference, a microRNA, an aptmer, a peptide or peptidomimetic, and an immunosuppressant.

13. The hydrogel of claim 1, further comprising a component of the extracellular matrix.

14. The hydrogel of claim 13, wherein the component of the extracellular matrix is collagen.

15. The hydrogel of claim 1, wherein the hyaluronan and gelatin are present at a ratio of about 5:5; a ratio of about 4:6; or a ratio of about 6:4.

16. The hydrogel of claim 1, wherein the gelatin is obtained from a human source.

17. A medical device comprising the hydrogel of claim 1.

18-20. (canceled)

21. A method for augmenting or repairing soft tissue, the method comprising:
(a) identifying a patient in need of tissue augmentation or repair; and
(b) administering to the patient the hydrogel of claim 1.

22. The method of claim 21, wherein the soft tissue is selected from the group consisting of sub-epithelial tissue, cartilage, liver, and neural tissue within the central or peripheral nervous systems.

23. The method of claim 22, wherein the sub-epithelial tissue is dermal tissue.

24. The method of claim 22, wherein the sub-epithelial tissue is tissue that has been damaged by trauma.

25. The method of claim 22, wherein the sub-epithelal tissue is tissue that has been damaged by disease.

26. The method of claim 25, wherein the disease is diabetes.

27. A method for delivering a bioactive agent to a patient, the method comprising:
(a) identifying a patient in need of treatment with the bioactive agent; and
(b) administering to the patient the hydrogel of claim 10.

28. A method of making a self-crosslinkable hydrogel composition, the method comprising:
(a) providing a first solution of partially oxidized hyaluronan;
(b) providing a second solution of gelatin;
(c) mixing the first solution of oxidized hyaluronan with the second solution of gelatin to obtain a third solution;
(d) allowing the hyaluronan and gelatin to cross-link in the third solution.

29. (canceled)
